(19) United States
(12) Patent Application Publication
(10) Pub. No.: US 2026/0014022 A1
(43) Pub. Date: Jan. 15, 2026

(54) ILLUMINATED OPHTHALMIC SURGICAL INSTRUMENT FOR PERFORMING GLAUCOMA TREATMENTS

(71) Applicant: Alcon Inc., Fribourg (CH)

(72) Inventors: Corey STEWART, Laguna Niguel, CA (US); Kevin Christopher Canzone, Coto de Caza, CA (US); Andrei DEEV, Pasadena, CA (US); Mikhail OVCHINNIKOV, Trabuco Canyon, CA (US); Keith Kiyoshi WATANABE, Irvine, CA (US)

(21) Appl. No.: 19/248,177

(22) Filed: Jun. 24, 2025

Related U.S. Application Data
(60) Provisional application No. 63/669,661, filed on Jul. 10, 2024.

(51) Int. Cl. A61F 9/008; A61B 90/20; G02B 21/00

(52) U.S. Cl. CPC A61F 9/008; A61B 90/20; G02B 21/0012; A61F 2009/00844; A61F 2009/00868; A61F 2009/00891; A61F 2009/00897

(57) ABSTRACT

A system includes a first light source configured to emit visible light according to one or more first parameters and a second light source configured to emit treatment light for altering patient tissue. A surgical instrument includes a distal portion configured to insert within an eye of a patient and an optical fiber conducts light from the first and second light sources to a tip of the distal portion. One or more sensors are configured to detect a state of the tip of the distal portion relative to the patient tissue. A controller is coupled to the one or more sensors, the first light source, and the second light source. The controller is configured to select values for the one or more first parameters according to the state of the tip of the distal portion relative to the patient tissue.

### CROSS-REFERENCE TO RELATED APPLICATIONS

This application claims priority to and benefit of U.S. Provisional Application No. 63/669,661, filed Jul. 10, 2024, which is hereby assigned to the assignee hereof and hereby expressly incorporated by reference in its entirety as if fully set forth below and for all applicable purposes.

### BACKGROUND

Glaucoma, a group of eye diseases affecting the retina and optic nerve, is one of the leading causes of blindness worldwide. Most forms of glaucoma result when the intraocular pressure (IOP) increases to pressures above normal for prolonged periods of time. IOP can increase due to high resistance to the drainage of the aqueous humor relative to its production. Left untreated, an elevated IOP causes irreversible damage to the optic nerve and retinal fibers resulting in a progressive, permanent loss of vision.

Glaucoma is often treated by inserting an instrument through the cornea in order to make an incision or place a shunt or incision in the anterior chamber to facilitate drainage of fluid from the anterior chamber. A shunt may be placed, for example, in the trabecular meshwork, Schlemm's canal, suprachoroidal space, or elsewhere. During the treatment, the surgeon will view the anterior chamber and the instrument through gonioscope or an ophthalmic microscope in order to place the incision or shunt at an appropriate location with the application of an appropriate amount of pressure.

It would be an advancement in the art to facilitate the performance of effective glaucoma treatments.

### SUMMARY

In certain embodiments, a system includes a first light source configured to emit visible light according to one or more first parameters. A second light source is configured to emit treatment light according to one or more second parameters in order to alter patient tissue. A surgical instrument includes a distal portion configured to insert within an eye of a patient. An optical fiber is coupled to the first light source and the second light source and conducts the visible light and the treatment light along the distal portion to be emitted from a tip of the distal portion. One or more sensors are configured to detect a state of the tip of the distal portion relative to the patient tissue. A controller is coupled to the one or more sensors, the first light source, and the second light source. The controller is configured to select values for the one or more first parameters according to the state of the tip of the distal portion relative to the patient tissue.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example operating environment for providing glaucoma treatments using an illuminated ophthalmic surgical instrument in accordance with certain embodiments.

FIG. 2 is cross-sectional view of the eye with an inserted illuminated ophthalmic surgical instrument in accordance with certain embodiments.

FIG. 3 is a side view of the illuminated ophthalmic surgical instrument and associated components for providing guidance during ophthalmic surgery in accordance with certain embodiments.

FIG. 4A is a schematic diagram of a light source for the illuminated ophthalmic surgical instrument in accordance with certain embodiments.

FIG. 4B is a schematic diagram of a light source and photodetector for the illuminated ophthalmic surgical instrument in accordance with certain embodiments.

FIG. 5A is cutaway cross-sectional view of the anterior chamber of the eye illustrating illumination of an incision site using the illuminated ophthalmic surgical instrument in accordance with certain embodiments.

FIG. 5B is cutaway cross-sectional view of the anterior chamber of the eye illustrating cutting of an incision using the illuminated ophthalmic surgical instrument in accordance with certain embodiments.

FIGS. 6A to 6D illustrate uses for illumination provided by the illuminated ophthalmic surgical instrument in accordance with certain embodiments.

FIG. 7 is a process flow diagram of a method for providing guidance during an ophthalmic surgery using the illuminated ophthalmic surgical instrument in accordance with certain embodiments.

FIG. 8 illustrates an example computing device that implements, at least partly, one or more functionalities for providing guidance during an ophthalmic surgery using the illuminated ophthalmic surgical instrument in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

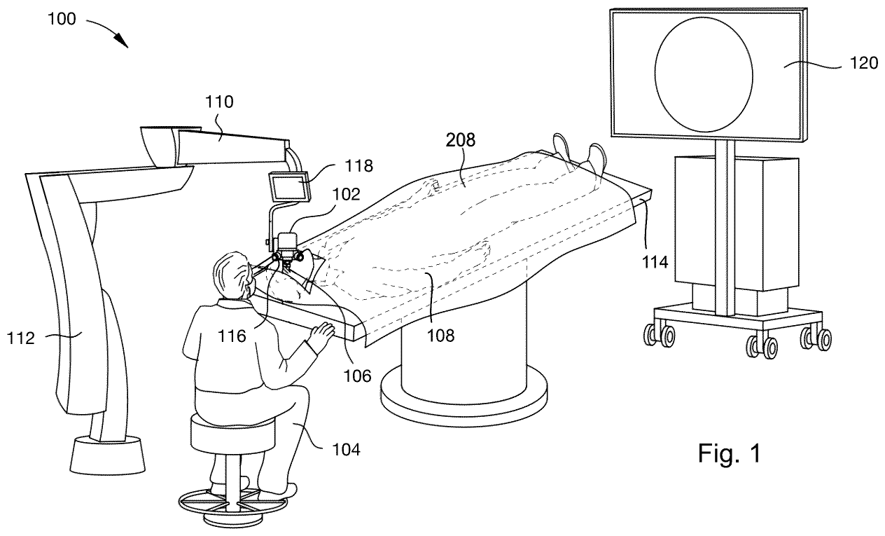

FIG. 1 illustrates an example system 100 in which an illuminated ophthalmic surgical instrument (hereinafter “the illuminated instrument”) may be used. The system 100 includes an ophthalmic microscope 102. A surgeon 104 uses the ophthalmic microscope 102 to visualize structures on and in an eye 106 of a medical patient 108 undergoing a surgery. The ophthalmic microscope 102 is supported on, in this illustration, an adjustable overhead arm 110 of a microscope support pedestal 112. The patient 108 may be supported on an operating table 114. The ophthalmic microscope 102 is movable with the overhead arm 110 in three dimensions so that the surgeon 104 can position the ophthalmic microscope 102 as desired with respect to the eye 106 of the patient 108.

In certain embodiments, the ophthalmic microscope 102 comprises a high resolution, high contrast stereo viewing surgical microscope. The ophthalmic microscope 102 will often include a monocular eyepiece 116 or binocular eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and broadband light source for capturing color (red, green, and blue) images, a multi-spectral imaging (MSI) device, and/or other type of imaging device. Digital images captured using the camera may be displayed on a display device within the ophthalmic microscope 102.

The ophthalmic microscope 102 may include two display devices viewable through binocular eyepieces 116 and that display images of the patient's eye 106 that are captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may be additionally or alternatively be displayed on one or more display devices. For example, the one or more display devices may include a display device 118 fastened to the supporting arm 110 above the ophthalmic microscope 102.

In order to relieve the surgeon 104 from the need to constantly look into the eye pieces 116 to obtain a stereoscopic view, the one or more display devices may include a display device 120 may be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device. The display device 120 may be mounted to a cart, a manually adjustable or robotic arm, or other manually or automatically adjustable support.

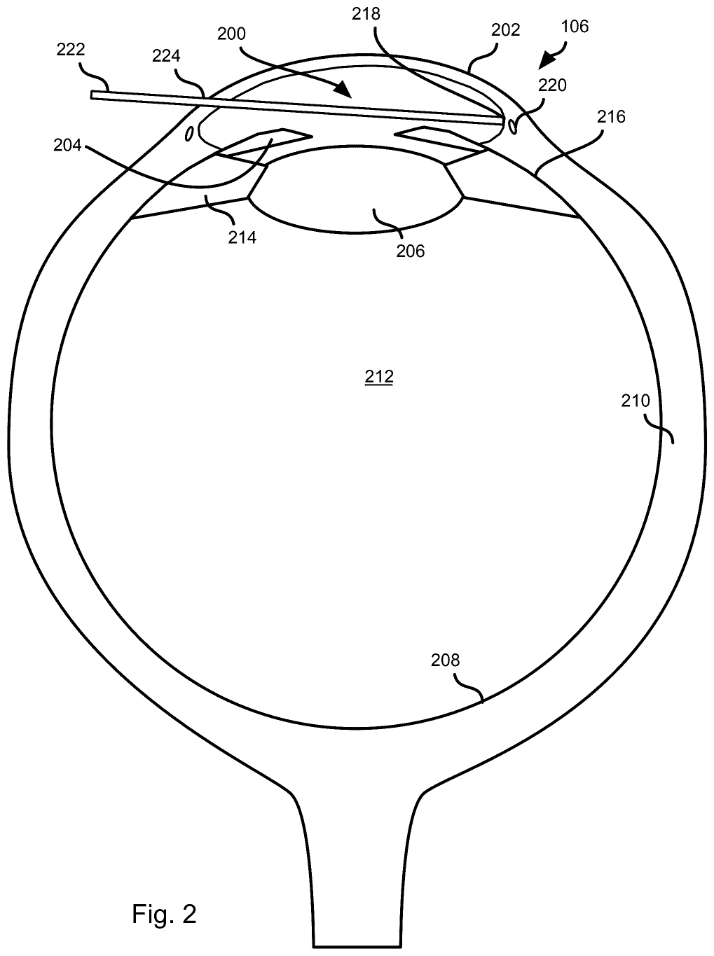

FIG. 2 is a diagram illustrating structures in and around anterior chamber 200 that are relevant to the treatment of glaucoma. The anterior chamber 200 of the eye is located behind the transparent and spherical cornea 202 through which light enters the eye. The iris 204 is a ring of muscles defining the pupil of the eye through which light passes. The crystalline lens 206 is located behind the pupil and, together with the cornea 202, focuses light onto the light sensitive cells of the retina 208. The retina 208 is formed on the interior of the globe 210 of the eye opposite the anterior chamber 200. The globe 210 of the eye between the lens 206 and the retina 208 is occupied by a transparent gel known as the vitreous 212.

The ciliary body 214 includes ligaments and muscles that connect the iris 204 and lens 206 to the choroid 216 of the eye. The muscles of the ciliary body 214 are responsible for altering the shape of the lens 206. The choroid 216 is a vascularized layer lining the globe 210 of the eye.

The ciliary body 214 produces the aqueous humor, which is the fluid that occupies the anterior chamber 200. The aqueous humor washes over the lens 206 and iris 204 and flows to the perimeter of the anterior chamber 200. The perimeter of the anterior chamber includes structures that, when functioning normally, allow the aqueous humor to drain. These structures include the trabecular meshwork 218 and Schlemm's canal 220. The trabecular meshwork 218 seems to act as a filter, limiting the outflow of aqueous humor and providing a back pressure that directly relates to IOP. Schlemm's canal 220 is located beyond the trabecular meshwork 218. Schlemm's canal 220 is fluidically coupled to collector channels (not shown) allowing aqueous humor to flow out of the anterior chamber 200.

Glaucoma may be treated by inserting the illustrated rod 222 into the anterior chamber 200, such as through an incision in the limbus 224 at the boundary between the cornea 202 and the sclera (white) of the eye. The rod 222 is then used to place an incision and possibly a shunt in one or more structures at the perimeter of the anterior chamber 200 to facilitate drainage of the aqueous humor. For example, an incision or shunt may be placed in the trabecular meshwork 218 to facilitate drainage into Schlemm's canal 220. In other approaches, a shunt extends from the anterior chamber into a suprachoroidal space between the choroid 216 and globe 210 of the eye.

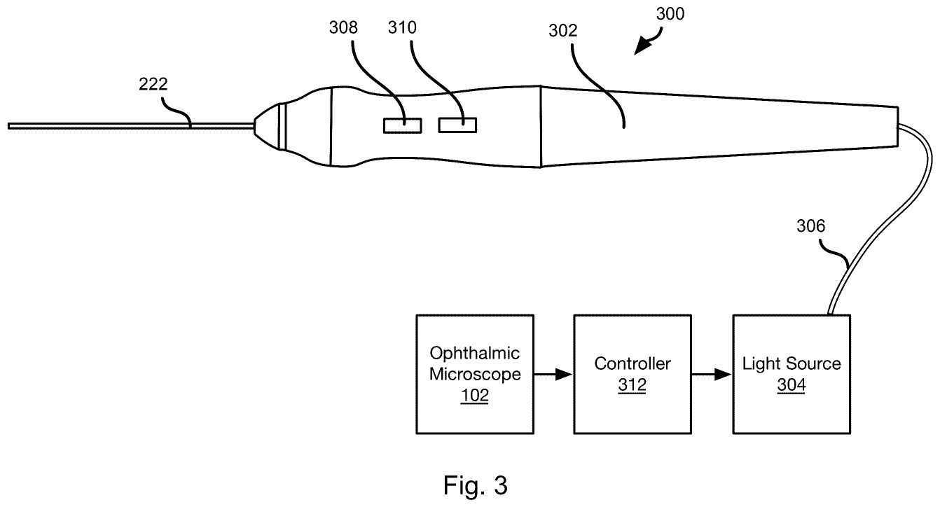

Referring to FIG. 3, an illuminated surgical instrument 300 may include the rod 222 and a handpiece 302 to which the rod 222 is mounted. The rod 222 is coupled through the handpiece to a light source 304, such as by an optical fiber 306. The light source 304 may be external to the handpiece 302 or mounted within the handpiece 302. The handpiece 302 is designed to be held in the hand of a surgeon 104 and may have one or more buttons 308, 310 or other interface elements mounted thereto and electrically coupled to the light source 304 to control operation of the light source 304.

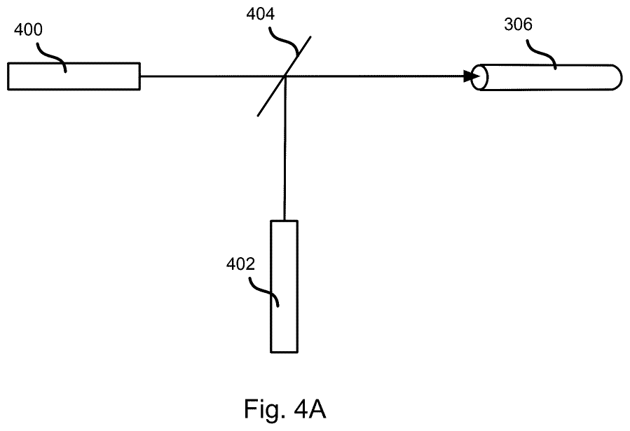

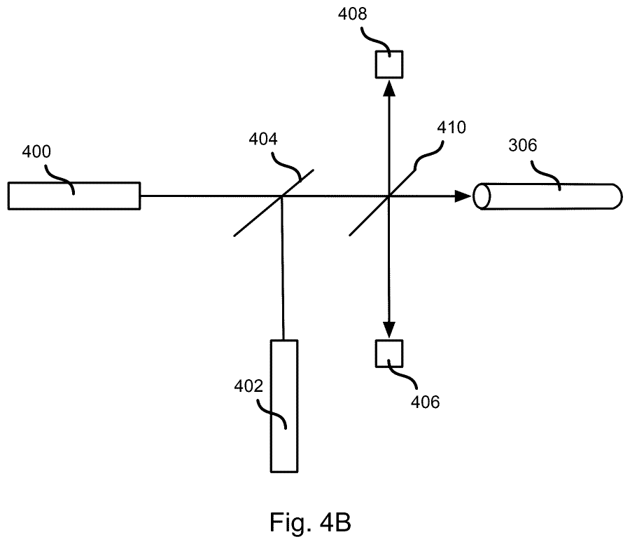

For example, referring to FIG. 4A while still referring to FIG. 3, the light source 304 may include a treatment light source 400 and an illumination light source 402. The treatment light source 400 may be embodied as a laser providing light of sufficient intensity to disintegrate tissue up to a prescribed distance from the tip of the rod 222. For example, the treatment light source 400 may be embodied as an infrared laser, such as an infrared laser diode. Parameters of the treatment light source 400 that may be controlled may include pulse energy, pulse duration and pulse frequency.

The illumination light source 402 emits light in the visible wavelength range (e.g., 380 to 700 nm). The illumination light source 402 may be implemented as one or more light emitting diodes (LED) emitting broadband light or a set of LEDs each with a peak intensity wavelength at a different point in the visible spectrum, such as red, green, blue, or other colors. Parameters of the illumination light source 402 that may be controlled may include intensity, pulse frequency (e.g., flashing frequency), pulse duration, color (e.g., different combinations of intensities for two or more LEDs having two or more different peak intensity wavelengths).

Light from the treatment light source 400 and illumination light source 402 may be made collinear and transmitted into the optical fiber 306, such as by means of a beam splitter 404. Note that in other embodiments, each of the treatment light source 400 and illumination light source 402 transmits light into a different optical fiber or into different cores of a multi-core optical fiber such that the beam splitter 404 is omitted.

Referring again to FIG. 3, a controller 312 may be coupled to the treatment light source 400 and illumination light source 402 and control values for the parameters of the treatment light source 400 and illumination light source 402 according to inputs received from an operator. For example, pressing of a first button 308 may invoke activation of the treatment light source 400 whereas pressing of a second button 310 may invoke activation of the illumination light source 402. The buttons 308, 310 are exemplary only and other interfaces may be used, such as a touch screen, voice commands, and/or camera capturing gestures, keyboard, pointing device and graphical user interface (GUI), or other type of interface. Interfaces capable of receiving more complex inputs may enable an operator to specify particular combinations of values for the parameters for each of the treatment light source 400 and illumination light source 402. In particular, the pulse energy, duration, and frequency may be selected to disintegrate a proscribed thickness of tissue, e.g., sufficient to create an incision through the trabecular meshwork to the Schlemm's canal, without causing damage to deeper tissue.

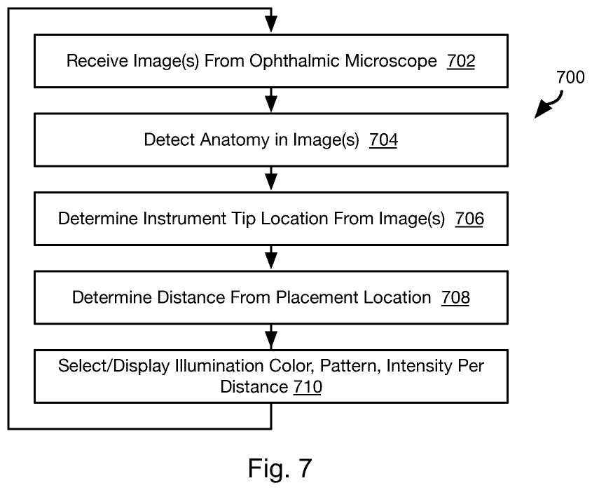

The controller 312 may be coupled to the ophthalmic microscope 102 and control the treatment light source 400 and/or the illumination light source 402 according to analysis of images received from the ophthalmic microscope 102 and possibly a treatment plan guiding a procedure being performed using the ophthalmic microscope. An example method for controlling the treatment light source 400 and/or the illumination light source 402 based on the images and possibly a treatment plan is described below with respect to FIG. 7.

Referring to FIG. 4B, in some embodiments, the illumination light source 402 may additionally include one or more photodetectors 406, 408. The photodetectors 406, 408 may be embodied as photodiodes. The photodetectors 406, 408 may include filters, such as filters filtering out light in the visible spectrum from the illumination light source 402 or filtering out light in the spectrum of the treatment light source 400, e.g., infrared.

The photodetector 406 may detect light reflected back by tissue of the patient's eye into the optical fiber 306 toward the treatment light source 400 and illumination light source 402. The photodetector 408 may detect a portion of the light emitted by one or both of the treatment light source 400 and illumination light source 402 that has not first been reflected from tissue of the patient's eye. For example, a beam splitter 410 may be positioned between the beam splitter 404 and the optical fiber 306 and direct a portion of the light that would go into the optical fiber 306 in the absence of the beam splitter 410 onto the photodetector 408 without first reflecting from the patient's eye. At least a portion of the light reflected back through the optical fiber 306 from tissue of the patient's eye may be directed by the beam splitter 410 onto the photodetector 406. The beam splitter 410 may be primarily transmissive such that only a small portion of the light incident on the beam splitter 410, e.g., between 1 and 10%, will be reflected onto the photodetectors 406, 408.

The controller 312 may receive the outputs of the photodetectors 406, 408 to detect a state of a tip of the rod 222 relative to the trabecular meshwork 218 or other tissue. For example, the controller 312 may estimate the distance between the tip of the rod 222 and tissue, such as the trabecular meshwork, based on the difference between the outputs of the photodetectors 406, 408, e.g., the output of photodetector 408 minus the output of photodetector 406, the output of photodetector 406 divided by the output of the photodetector 408, a weighted output of the photodetector 408 minus the weighted output of the photodetector 406, or some other function of the outputs of the photodetectors 406, 408. For example, detector 406 may measure a waveform or reflected optical pulse in the time domain, which has some properties such as amplitude and pulse width. When the amplitude falls below a minimum threshold, which may be zero, and/or a reflected pulse is below a minimum threshold or is undetectable, the tip of the rod 222 may be determined to have made contact and the treatment light source 400 may be activated to create an incision.

For example, the lower the difference between the output of the photodetector 406 and the output of the photodetector 408 the greater the reflection and therefore the closer the tip of the rod 222 to tissue of the eye. The controller 312 may select values for one or more parameters, such as at least two parameters, of light emitted by the illumination light source 402 based on the difference. For example, a pulse frequency may increase with decreasing of the difference. In another example, an intensity may increase with decreasing of the difference. In yet another example, a color may transition from a first color to a second color with decreasing of the difference. In some embodiments, in response to the difference reaching a predefined value, or range of values, the controller 312 may alter the color of light emitted by the illumination light source 402, e.g., change from white to green, indicating that the tip of the rod 222 is at an appropriate distance from the tissue at which the illumination light source 402 may be activated to create an incision.

Using measurements from the photodetectors 406, 408 to provide guidance to the surgeon 104 has the advantage of not requiring the use of an ophthalmic microscope. For example, a surgeon may use a gonioscope to visualize the anterior chamber 200. The gonioscope may further define channels to guide the rod 222 into the anterior chamber 200.

As described below with respect to FIG. 7, an ophthalmic microscope 102 may be used in some embodiments to enable guidance to the surgeon 104 using light from the illumination light source 402. The photodetectors 406, 408 and ophthalmic microscope 102 are just examples of sensors that may be used to sense the position of the tip of the rod 222 relative to the trabecular meshwork 218 or other tissue. Ultrasonic measurement may be used to determine the location of the tip of the rod 222 relative to the trabecular meshwork 218 or other tissue. Two or more visible light cameras having the anterior chamber 200 in the field of view thereof may be used to determine the location of the tip of the rod 222 without being incorporated into an ophthalmic microscope 102. In some embodiments, a camera, which may be disposable, is housed in the handpiece 302 or elsewhere and images the anterior chamber 200 using light received through an optical fiber passing through or along the rod 222, which may be the optical fiber 306 or a different optical fiber.

The implementations of the light source 304 described above with respect to FIGS. 4A and 4B is exemplary only. The light source 304 and the surgical instrument 300 coupled to the light source 304 may be implemented as described in any of the following references, all of which are hereby incorporated herein by reference in their entirety:
U.S. patent application Ser. No. 16/003,175, filed Jun. 18, 2018, and entitled BIREFRINGENT LENS FOR LASER BEAM DELIVERY;
U.S. patent application Ser. No. 16/219,139 filed Dec. 13, 2018, and entitled ULTRAVIOLET LASER VITRECTOMY PROBE; and
U.S. patent application Ser. No. 17/662,148, filed May 5, 2022, and entitled SURGICAL LASER SYSTEM WITH ILLUMINATION.

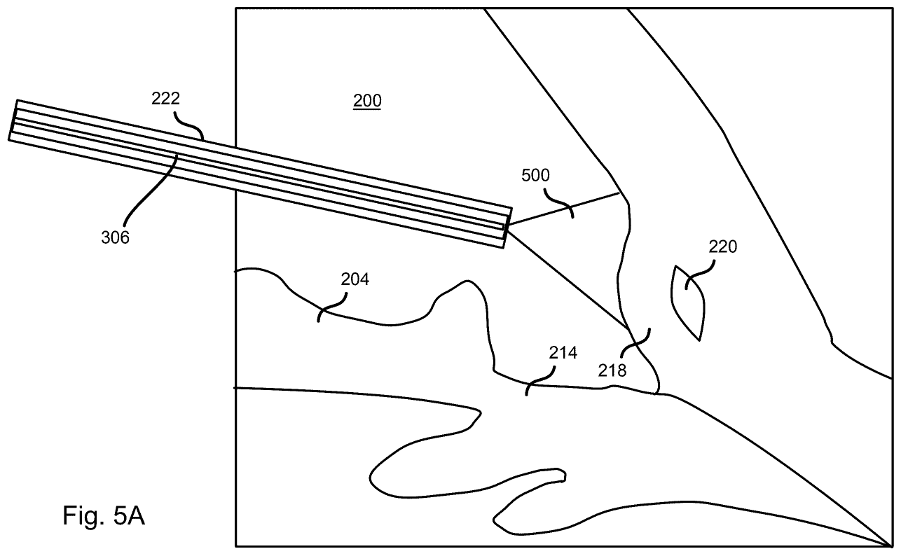

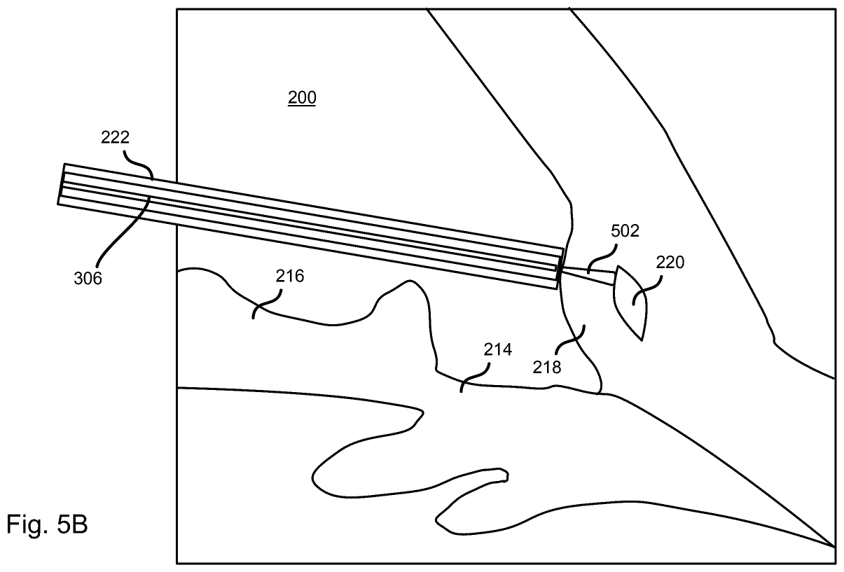

Referring to FIG. 5A, in use, the illumination light source 402 may be activated with the treatment light source 400 being deactivated. The optical fiber 306 extends through or along a distal portion of a surgical instrument, such as through the rod 222, or along the rod 222, of the surgical instrument 300. The optical fiber 306 emits a beam 500 that projects beyond a tip of the rod 222. Where the illumination light source 402 is a broadband and non-coherent light source, the beam 500 may diffract substantially and illuminate a large area of the anterior chamber 200, e.g., an illuminated spot diameter at least 5 times, 10 times, or 15 times larger than the diameter of the optical fiber 306 at the tip of the rod 222 when at least 3 mm away from the illuminated spot. This illumination, alone or in combination with light from the ophthalmic microscope 102, illuminates the area to be treated, such as the trabecular meshwork 218. This illumination enables the surgeon 104 to place the tip of the rod 222 at an appropriate location.

Referring to FIG. 5B, the surgeon 104 may then activate the treatment light source 400 to administer a treatment. The light from the treatment light source 400 passes through the optical fiber 306, resulting in a beam 502 emitted beyond a tip of the rod 222. The illumination light source 402 may be deactivated when the treatment light source 400 is activated or may remain activated. In some embodiments, the treatment light source 400 is pulsed and pulses of light from the illumination light source 402 may be interleaved with pulses of light from the treatment light source 400. For example, activating of the illumination light source 402 may enable the surgeon 104 to view bubbles or other visible effects of the operation of the treatment light source 400, which itself is not visible.

The treatment light source 400 may be a coherent or non-coherent light source. Where the treatment light source 400 is a coherent light source, the beam 502 may be much narrower than the beam 500 of the illumination light source 402, such as having a numerical aperture less than 25 percent, less than 10 percent, or less than 5 percent, of the numeral aperture of the beam 500. The tip of the rod 222 may have lens fastened thereto and positioned to focus light from the optical fiber 306 at a particular depth and limit the depth at which the beam 502 is able to disintegrate tissue. Such a lens may further facilitate broadening a spot in the anterior chamber 200 illuminated by the beam 500 when the tip of the rod 222 is within the anterior chamber 200 offset from the tissue of the eye by a distance greater than a distance between the tip of the rod 222 and the focal point of the lens.

The beam 502 is effective to disintegrate the portion of the trabecular meshwork 218 between the tip of the rod 222 and Shlemm's canal 220 thereby creating a passage enabling drainage of the aqueous humor from the anterior chamber 200 into Schlemm's canal. The values for the parameters controlling the treatment light source 400 may be selected according to a treatment plan, the values being selected to provide an appropriate depth of disintegration of the trabecular meshwork 218. The process illustrated in FIGS. 5A and 5B may be repeated at various points along the perimeter of the anterior chamber 200 in order to create sufficient passages to lower (e.g., reduce) IOP in the anterior chamber 200 and the vitreous 212. The multiple passages may be created using a single incision through the limbus 224 or multiple incisions through the limbus 224.

In some embodiments, the treatment light source 400 is not used and an incision is formed using a cutting edge, shunt, or other structure secured to the rod 222. In such embodiments, the rod 222 may still include the optical fiber 306 to conduct light from the light source 402 to guide the surgeon 104 to place an incision at an appropriate location.

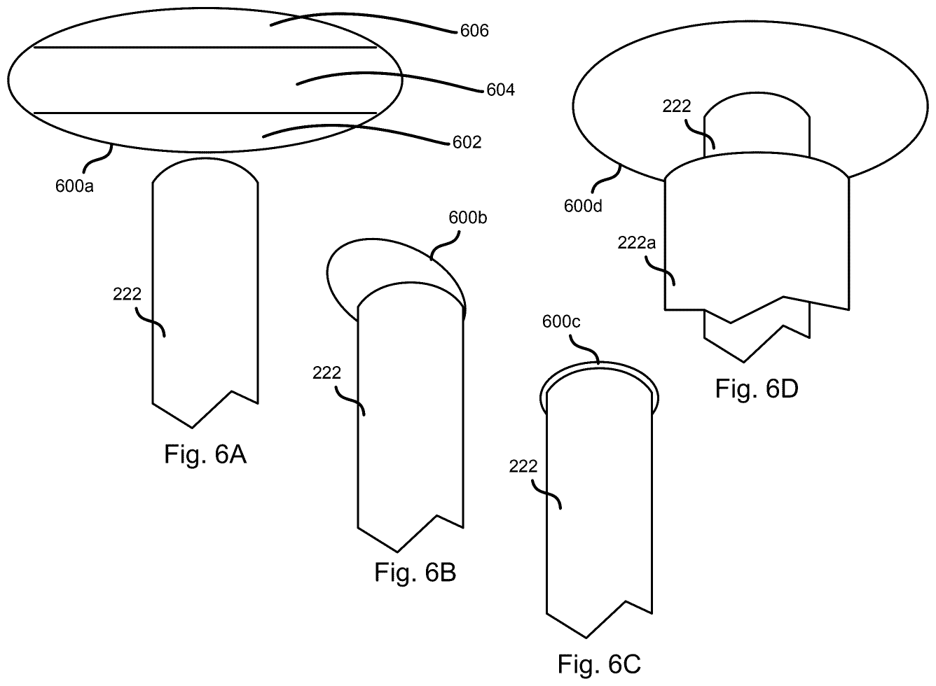

Referring to FIGS. 6A to 6D, in some embodiments, illumination provided with light from the illumination light source 402 may provide guidance to a surgeon 104 passively, i.e., without computation and outputs by the controller 312. The guidance illustrated with respect to FIGS. 6A to 6D may be obtained with a controller 312 requiring no more logic than is required to activate the treatment light source 400 and illumination light source 402 responsive to inputs from the surgeon 104, such as by pressing the buttons 308, 310. The guidance illustrated with respect to FIGS. 6A to 6D may be obtained without using an ophthalmic microscope 102. For example, a surgeon may use a gonioscope to visualize the anterior chamber 200 and still achieve the benefits described below with respect to FIGS. 6A to 6D.

For example, referring to FIG. 6A, with the tip of the rod 222 offset from the trabecular meshwork 218, e.g., at least 1 mm, at least 2 mm, or at least 4 mm, the light emitted from the tip of the rod 222 will illuminate a spot 600a on the trabecular meshwork 218. The perimeter of the anterior chamber 200 has different bands that are different in appearance. These bands include, moving outward from the ciliary body 214, the ciliary body band 602, pigmented trabecular meshwork 604, and the non-pigmented trabecular meshwork 606. The Schlemm's canal 220 is located behind the pigmented trabecular meshwork 604. Accordingly, using the visibility provided by the spot 600a, the surgeon 104 may move the tip of the rod 222 into alignment with, and possibly in contact with, the pigmented trabecular meshwork 604 prior to activating the treatment light source 400.

In addition to the visibility provided by illumination of the spot 600a, the illumination light source 402 may be strobed (e.g., pulsed at a pulse frequency between 10 to 12 Hz with pulse durations of between 10 and 50 milliseconds. Strobing may enhance visibility by reducing blurring due to movement. For example, light from the treatment light source 400 may vaporize tissue or fluid resulting in rapidly forming and collapsing bubbles. Strobing of the illumination light source 402 may help avoid blurring due to bubbles.

Referring to FIG. 6B, in another example, the light from the illumination light source 402 may illuminate a spot 600b on the perimeter of the anterior chamber 200. When the rod 222 is not normal to the spot 600b, the spot 600b will extend to one side of the rod 222 thereby communicating to the surgeon the need to adjust the angle of the rod 222 and the direction in which to adjust the rod 222.

Referring to FIG. 6C, when the tip of the rod 222 is approximately (e.g., within 0.5 degrees) normal to a point of contact with the trabecular meshwork 218 and is in contact with the trabecular meshwork 218, the illuminated spot 600c will be at a minimum. The illuminated spot 600c may be either not visible to the surgeon or include only scattered light transmitted out through the tissue of the trabecular meshwork 218. The surgeon will therefore know that further movement toward the trabecular meshwork 218 is not required and that the treatment light source 400 may be activated to create a passage through the trabecular meshwork 218.

Referring to FIG. 6D, in some embodiments, multiple optical fibers 306 or a multi-core optical fiber may be used. For example, the rod 222 may be an inner rod as described above. An outer layer 222a may surround the rod 222 and include one or more additional fibers or one or more layers of a multi-core fiber. The rod 222 may extend distally of the outer layer 222a and light may be transmitted from the outer layer 222a even when the tip of the rod 222 is located adjacent (e.g., within 1 mm) of the trabecular meshwork 218. Accordingly, a surgeon will still have sufficient light in illuminated spot 600d to facilitate correct placement of the tip of the rod 222. In some embodiments, transmission of light to through the rod 222 and transmission of light between the outer layer 222a and the rod 222 may be independently controlled.

FIG. 7 illustrates a method 700 that may be executed by the controller 312 in order to provide active guidance to the surgeon 104 using illumination transmitted through the rod 222 based on a state of a tip of the rod 222 relative to the trabecular meshwork 218 or other tissue. The state may correspond to a distance from the trabecular meshwork 218 and a vertical offset from a desired vertical location along the trabecular meshwork, where the vertical direction is defined as parallel to the optical axis of the eye. The method 700 may presume usage of an ophthalmic microscope 102 capable of capturing monocular or stereoscopic images of the anterior chamber or some other imaging modality. However, images from cameras other than those incorporated into an ophthalmic microscope may be used in a like manner to images from the ophthalmic microscope 102 as described below.

The method 700 may include receiving, at step 702, one or more images from the ophthalmic microscope 102 and detecting, at step 704, anatomy in the one or more images. As noted above, the trabecular meshwork 218 has visually distinct bands. Accordingly, step 704 may include identifying these bands and a location thereof. Step 704 may be implemented using a machine learning model trained to perform this task, machine vision algorithm, or other approach for computationally recognizing features in monocular or stereoscopic images. Step 704 may include registering the one or more images with respect to one or more corresponding reference images of the same eye as the one or more images and having labeled representations of the bands.

The method 700 may include determining, at step 706, a location of a tip of a distal portion of an instrument from the one or more images, such as tip of the rod 222 of an illuminated instrument 300. Step 706 may include identifying a representation of the tip of the rod 222 in each of the one or more images. Step 706 may be implemented using a machine learning model trained to perform this task, a machine vision algorithm, or other approach for computationally recognizing features in monocular or stereoscopic images. Where the one or more images include two stereoscopic images, the three-dimensional location of the tip of the rod 222 may be readily determined using known techniques and properties of the digital ophthalmic microscope, such as the direct linear transform (DLT).

The method 700 may include determining, at step 708, a distance from the location of the tip of the rod 222 to a placement location. The location may be defined with respect to two or more dimensions. For example, the anterior chamber 200 may define a vertical direction parallel to the optical axis of the eye, a radial direction extending outwardly perpendicular to the optical axis and a circumferential direction defined as a movement or orientation along a circle centered on the optical axis. For glaucoma surgery, the circumferential direction is the least critical of these directions. Accordingly, step 706 may include identifying the location of the tip of the rod 222 in only the radial and vertical directions.

The placement location may be defined in three dimensions or in just two dimensions, i.e., the radial and vertical directions. For example, the placement location may be defined in a treatment plan uploaded to the controller 312 and/or ophthalmic microscope 102. The distance may therefore be determined at step 708 by determining a difference between a coordinate of the tip of the rod 222 and a corresponding coordinate of the placement location for the two dimensions or all three dimensions.

The method 700 may include selecting, at step 710, values for one or more parameters, such as at least two parameters, for light generated by the illumination light source 402 based on the distance and causing the illumination light source 402 to output light corresponding to the one or more parameters. These parameters may include some or all of color, a blinking pattern, intensity, or other parameters. For example, there may be a number of scenarios, each of which has a clearly recognizable set of values for the parameters associated therewith, such as those listed in Table 1. Note that a set of values for the parameters corresponding to placement that is too close radially is not defined in some embodiments since the tip of the rod 222 would be obscured. However, in some embodiments, such as that shown in FIG. 6D, a set of values for the parameters corresponding to placement that is too close to the trabecular meshwork 218 may also be defined. As used herein “on target” means within a predefined region, such as within a predefined tolerance of the placement location.

TABLE 1
Scenarios having corresponding values for illumination parameters.

| | |
|---|---|
| 1 | Too Far Radially, Too Low Vertically |
| 2 | On Target Radially Too Low Vertically |
| 3 | Too Far Radially, Too High Vertically |
| 4 | On Target Radially, Too High Vertically |
| 5 | Too Far Radially, On Target Vertically |
| 6 | On Target |

The values for the parameters for a given scenario may vary in magnitude. For example, let red indicate too low vertically, yellow indicate too high vertically, and green indicate on target vertically. A higher intensity of red light may indicate greater displacement above the placement location relative to a lower intensity. A higher intensity of yellow light may indicate greater displacement below the placement location than a lower intensity. Let another parameter, such as flashing frequency indicate radial proximity, e.g., flashing at a frequency that increases with proximity to the trabecular meshwork 218. Accordingly, red light flashing at a first frequency may indicate that the tip of the rod 222 is at a first radial distance relative to the trabecular meshwork 218 and a red light flashing at a second frequency that is higher than the first frequency may indicate that the tip of the rod 222 is at a second radial distance relative to the trabecular meshwork 218 that is closer than the first radial distance. A solid red light may indicate that the tip of the rod 222 is on target along the radial direction but too low vertically. Green and yellow lights may blink based on radial position in the same manner for on-target or too high vertical positioning, respectively.

In another example, blinking in alternate colors, e.g., two different colors may be used. For example, let red indicate too low vertically, yellow indicate too high vertically, and green indicate on target vertically. Let the intensity of blue correspond to distance from the trabecular meshwork. In this example, alternating blue and red flashes indicate that the tip of the rod 222 is too far away radially and too low vertically, alternating blue and yellow indicates too far away radially and too high vertically, and alternating green and blue indicates on target vertically and too far away radially. Alternating green and blue with the intensity of the blue fading to green (or some other color) indicates movement toward the trabecular meshwork with the proper vertical alignment. When the light becomes a constant green, the correct radial and vertical position has been achieved. Likewise, a solid red or yellow light indicates on target positioning in the radial direction with a vertical position that is too low or too high, respectively.

The above-described examples are exemplary only. Any combinations of visually distinguishable colors may be used in place of those listed above. Any combination of values for any of the parameters for the light sources 402 may likewise be used to indicate displacement of the tip of the rod 222 relative to the placement location.

Although the foregoing description is focused on treatments for glaucoma, other types of treatments may be advantageously use an illuminated ophthalmic surgical instrument. For example, phacoemulsification is the breakup and removal of the lens 206, which is an initial step to the placement of an intraocular lens (IOL) for treating cataracts or other conditions. A phacoemulsification tool may use light from the treatment light source 400, or some other type of cutting tool, to disintegrate the lens 206 along with a tube coupled to a vacuum for removing the disintegrated lens 206. The tube may be incorporated into the rod 222 or extend along the rod 222 to the tip of the rod or near the tip of the rod (e.g., within 0.1 mm). Light from the illumination light source 402 may be used to illuminate the lens 206 prior to and/or during phacoemulsification. Strobing of the illumination light source 402 may facilitate visualization where bubbles are rapidly forming. The location of the tip of the rod 222 may be monitored as described above. The values for parameters controlling the generation of light by the illumination light source 402 may be used to provide guidance, such as providing a color change, flashing pattern, or other visible cue warning the surgeon 104 when the tip of the rod 222 is at, or within a threshold distance of, the capsular bag containing the lens 206 in order to avoid bag rupture.

In another example, guidance may be provide when performing a vitrectomy to remove the vitreous 212. The rod 222 may incorporate the optical fiber 306 as well as a vacuum tube as described above. The rod 222 may include a vitreous cutter. In embodiments where light from a treatment light source 400 is used to disintegrate the vitreous 212, the vitreous cutter may be omitted. Light from the illumination light source 402 may be used to illuminate the lens 206 prior to and/or during vitrectomy. Strobing of the illumination light source 402 may facilitate visualization where bubbles are rapidly forming. The values for parameters controlling the generation of light by the illumination light source 402 may be used to provide guidance, such as providing a color change, flashing pattern, or other visible cue warning the surgeon 104 when the tip of the rod 222 is at, or within a threshold distance of, the retina 208 or choroid 216.

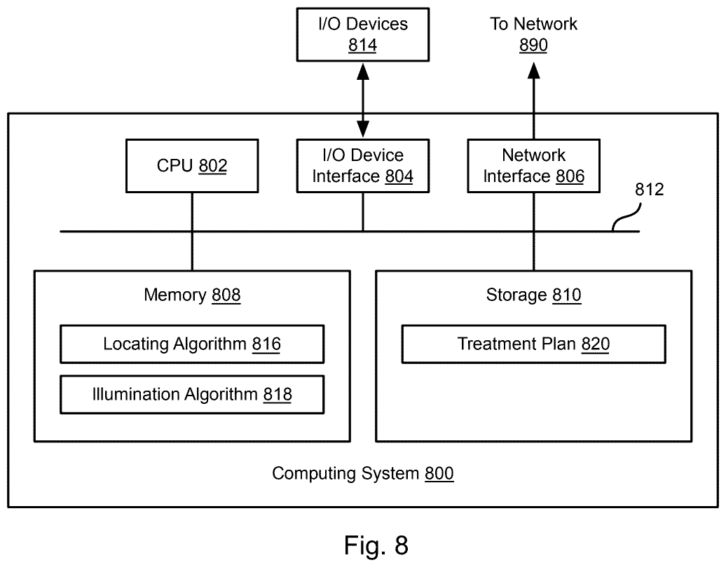

FIG. 8 illustrates an example computing system 800. The controller 312 may have some or all of the attributes of the computing system 800. The ophthalmic microscope 102 and the display device 120 may likewise incorporate a computing device having some or all of the attributes of the computing system 800.

As shown, computing system 800 includes a central processing unit (CPU) 802, one or more input/output (I/O) device interfaces 804, which may allow for the connection of various I/O devices 814 (e.g., keyboards, displays, mouse devices, pen input, etc.) to computing system 800, network interface 806 through which computing system 800 is connected to network 890, a memory 808, storage 810, and an interconnect 812.

CPU 802 may retrieve and execute programming instructions stored in the memory 808. Similarly, CPU 802 may retrieve and store application data residing in the memory 808. The interconnect 812 transmits programming instructions and application data, among CPU 802, I/O device interface 804, network interface 806, memory 808, and storage 810. CPU 802 is included to be representative of a single CPU, multiple CPUs, a single CPU having multiple processing cores, and the like.

Memory 808 is representative of a volatile memory, such as a random access memory, and/or a nonvolatile memory, such as nonvolatile random access memory, phase change random access memory, or the like. As shown, memory 808 may store executable code implementing a locating algorithm 816 defining executable code, machine learning models, or other instructions for identifying anatomy at step 704 and the tip of the rod 222 at step 706. The memory 808 may store executable code 818 defining an illumination algorithm defining the selection of parameters for the illumination light source 402 as described above with respect to steps 708 and 710.

Storage 810 may be non-volatile memory, such as a disk drive, solid state drive, or a collection of storage devices distributed across multiple storage systems. Storage 810 may optionally store a treatment plan 820. The treatment plan may include one or more labeled reference images to facilitate detection of anatomy at step 704, define one or more placement locations, values for parameters for activating the treatment light source 400, or other information to facilitate administration of glaucoma treatments.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system comprising:
a first light source configured to emit visible light according to one or more first parameters;
a second light source configured to emit treatment light according to one or more second parameters in order to alter patient tissue;
a surgical instrument including a distal portion configured to insert within an eye of a patient;
an optical fiber coupled to the first light source and the second light source and conducting the visible light and the treatment light along the distal portion to be emitted from a tip of the distal portion;
one or more sensors configured to detect a state of the tip of the distal portion relative to the patient tissue; and
a controller coupled to the one or more sensors, the first light source, and the second light source, the controller configured to select values for the one or more first parameters according to the state of the tip of the distal portion relative to the patient tissue.

2. The system of claim 1, wherein the one or more first parameters include at least one of color, intensity, pulse frequency, and pulse duration.

3. The system of claim 1, wherein the one or more first parameters include at least two of color, intensity, pulse frequency, and pulse duration.

4. The system of claim 1, wherein the state of the tip of the distal portion includes a distance of the tip of the distal portion from the patient tissue.

5. The system of claim 1, wherein the state of the tip of the distal portion includes a location of the tip of the distal portion along an optical axis of the eye of the patient.

6. The system of claim 1, wherein the state of the tip of the distal portion includes a distance of the tip of the distal portion from a region within the eye of the patient.

7. The system of claim 1, wherein the state of the tip of the distal portion includes a distance of the tip of the distal portion from a region within an anterior chamber of the eye of the patient located over Schlemm's canal.

8. The system of claim 1, wherein the one or more sensors include a first photodetector configured to sense a first portion of the visible light that is not reflected from the patient tissue and a second photodetector configured to sense a second portion of the visible light that is reflected from the patient tissue.

9. The system of claim 8, wherein the controller is configured to select the values for the one or more first parameters according to a difference between an output of the first photodetector and an output of the second photodetector.

10. The system of claim 1, wherein:
the one or more sensors include one or more cameras; and
the controller is configured to:
receive one or more images from the one or more cameras;
identify a location of anatomy in the one or more images;
identify a location of the tip of the distal portion in the one or more images; and
select the values for the one or more first parameters based on the location of the anatomy and the location of the tip of the distal portion.

11. A method comprising:
inserting a distal portion of a surgical instrument into an eye of a patient, the distal portion including an optical fiber;
transmitting first light from a first light source through the optical fiber and emitting the first light from the optical fiber onto tissue of the eye of the patient, the first light being visible light; and
transmitting second light from a second light source through the optical fiber onto the tissue to create an incision facilitating reduction of intraocular pressure.

12. The method of claim 11, wherein the tissue is a trabecular meshwork of the eye.

13. The method of claim 11, further comprising:
selecting, by a controller coupled to the first light source and the second light source, first values for one or more first parameters, the first light being generated according to the one or more first parameters;
detecting, by one or more sensors coupled to the controller, a state of a tip of the distal portion relative to the tissue;
selecting, by the controller, one or more second values for the one or more first parameters according to the state;
illuminating, with the first light source, the tissue with third light generated according to the one or more second values for the one or more first parameters.

14. The method of claim 13, wherein the one or more first parameters include at least one of color, intensity, pulse frequency, and pulse duration.

15. The method of claim 13, wherein the state of the tip of the distal portion includes a distance of the tip of the distal portion from the tissue.

16. The method of claim 13, wherein the state of the tip of the distal portion includes a location of the tip of the distal portion along an optical axis of the eye of the patient.

17. The method of claim 13, wherein the state of the tip of the distal portion includes a distance of the tip of the distal portion from a region within an anterior chamber of the eye of the patient located over Schlemm's canal.

18. The method of claim 13, wherein the one or more sensors include a first photodetector configured to sense a first portion of the first light that is not reflected from the tissue and a second photodetector configured to sense a second portion of the first light that is reflected from the tissue.

19. The method of claim 18, further comprising selecting, by the controller, the one or more first parameters according to a difference between an output of the first photodetector and an output of the second photodetector.

20. The method of claim 13, wherein:
the one or more sensors include one or more cameras; and
the method further comprises:
receiving, by the controller, one or more images from the one or more cameras;
identifying, by the controller, a location of anatomy in the one or more images;
identifying, by the controller, a location of the tip of the distal portion in the one or more images; and
selecting, by the controller, the one or more second values for the one or more first parameters based on the location of the anatomy and the location of the tip of the distal portion.
